(19) United States
(12) Patent Application Publication
(10) Pub. No.: US 2013/0237705 A1
(43) Pub. Date: Sep. 12, 2013

(54) METHOD FOR MANUFACTURE OF 2-OXOIMIDAZOLIDINES

(71) Applicants: John R. Peterson, Chardon, OH (US); Christopher M. Yost, Ayr (CA)

(72) Inventors: John R. Peterson, Chardon, OH (US); Christopher M. Yost, Ayr (CA)

(21) Appl. No.: 13/854,444

(22) Filed: Apr. 1, 2013

Related U.S. Application Data
(63) Continuation-in-part of application No. 12/714,614, filed on Mar. 1, 2010, now Pat. No. 8,431,699.

(51) Int. Cl. C07D 233/36; C07D 405/14

(52) U.S. Cl. CPC C07D 233/36; C07D 405/14
USPC 544/370

(57) ABSTRACT

A method for the manufacture of 2-oxoimidazolidines comprising the steps of converting an isocyanate and an amine to a urea, and then performing a ring closure of the urea to yield the 2-oxoimidazolidine is disclosed. The 2-oxoimidazolidines produced may then be used in the production of Pramiconazole and other structurally related compounds.

This application is a continuation-in-part and claims priority to U.S. Ser. No. 12/714,614, entitled METHOD FOR MANUFACTURE OF 2-OXOIMIDAZOLIDINES, filed Mar. 1, 2010 and is incorporated herein by reference.

### I. BACKGROUND

Pramiconazole, a triazole antifungal compound, may be used for the treatment of acute and chronic seborrheic dermatitis and other fungal skin infections in humans. The structure of Pramiconazole is provided below in Structure I:

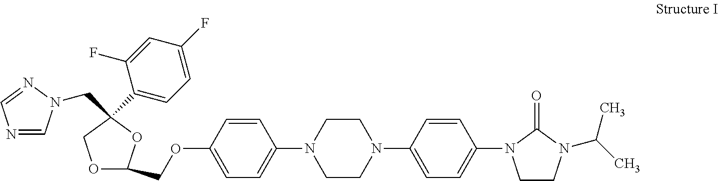 [SMILES: CC(C)N1CCN(c2ccc(N3CCN(c4ccc(OC[C@H]5OC[C@](Cn6cncn6)(c6ccc(F)cc6F)O5)cc4)CC3)cc2)C1=O]

Pramiconazole may be produced from 2-oxoimidazolidine, which is shown below in Structure II in a generalized chemical structure. The 2-oxoimidazolidine ring within the Pramiconazole is one of two requisite structural components for providing anti-fungal activity.

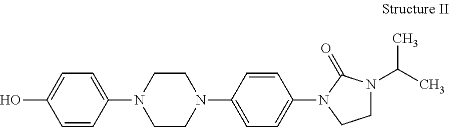 [SMILES: CC(C)N1CCN(c2ccc(N3CCN(c4ccc(O)cc4)CC3)cc2)C1=O]

As a result of the favorable clinical indications of Pramiconazole and structurally related drugs, there may be an interest in methods that provide 2-oxoimidazolidine, especially those methods that may be more cost effective relative to the prior art.

### II. SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

Accordingly, described herein is a method for the preparation of a 2-oxoimidazolidine comprising the steps of: (i) converting an isocyanate and an amine to a urea; and (ii) performing a ring closure of the urea to yield 2-oxoimidazolidine.

To the accomplishment of the foregoing and related ends, the following description and annexed drawings set forth certain illustrative aspects and implementations. These are indicative of but a few of the various ways in which one or more aspects may be employed. Other aspects, advantages and novel features of the disclosure will become apparent from the following detailed description when considered in conjunction with the annexed drawings.

### III. BRIEF DESCRIPTION OF THE FIGURES

What is disclosed herein may take physical form in certain parts and arrangement of parts, and will be described in detail in this specification and illustrated in the accompanying drawings which form a part hereof and wherein:

FIG. 1 provides a series of chemical reactions, which are disclosed herein.

### IV. DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

The word “exemplary” is used herein to mean serving as an example, instance or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. Of course, those skilled in the art will recognize many modifications may be made to this configuration without departing from the scope or spirit of the claimed subject matter.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure.

In addition, while a particular feature of the disclosure may have been disclosed with respect to only one of several implementations, such feature may be combined with one or more other features of the other implementations as may be desired and advantageous for any given or particular application. Furthermore, to the extent that the terms “includes,” “having,” “has,” “with,” or variants thereof are used in either the detailed description or the claims, such terms are intended to be inclusive in a manner similar to the term “comprising.”

As referred to herein, the terms “amine” and “amino” represent organic compounds and functional groups that contain a nitrogen atom to which two substituents and a hydrogen atom are bonded. Useful examples of amine may include, but are not limited to, methylamine, N,N-dimethylamine, ethylamine, N,N-diethylamine, N-ethyl-N-methylamine, 1-methylethylamine, N,N-di-(1-methylethyl)amine, N-(2-hydroxyethyl)-N-(1-methylethyl)amine, n-propylamine, N,N-di-n-propylamine, and n-butylamine. Useful examples of amino may include, but are not limited to, methylamino, dimethylamino, ethylamino, diethylamino, 1-methylethylamino, and di-(1-methylethyl)amino.

As referred to herein, the term “chiral” refers to a compound that contains one or more carbon atoms, the chiral center, to which four different groups are bonded. The spatial orientation of the groups attached to a chiral carbon center may be defined in absolute terms as either R or S, wherein the priority assignment of each substituent may be defined by Cahn-Ingold-Prelog sequence rules.

As referred to herein, the terms “cis” and “trans” refers to the spatial orientation of two groups attached to a ring or a double bond. The spatial orientation of these groups may be defined as being cis whenever the groups are attached to the same face of the ring or double bond, and as being trans whenever the groups are attached to opposite faces of the ring or double bond.

As referred to herein, the term “enantiomer” refers to the three-dimensional stereo-relationship between two chiral molecules that are minor image compounds. An enantiomer contains one or more chiral centers that impart a unique three-dimensional structure to the compound. Two enantiomers have identical physical properties with the exception of being able to rotate plane polarized light in equal amounts, but in opposite directions.

As referred to herein, the term “intermolecular” refers to a process or characteristic between the structures of two or more molecules.

As referred to herein, the term “intramolecular” refers to a process or characteristic within the structure of an individual molecule.

As referred to herein, the term “leaving group” or “L” refers to a group or atom that is displaced from the carbon atom to which it was attached by a nucleophile during a nucleophilic substitution reaction or by a base during an elimination reaction. Useful examples of a leaving group may include, but are not limited to, chloro, bromo, iodo, and hydroxy; or L is leaving group of formula —OR₈ wherein R₈ is a radical selected from the group that may include, but is not limited to, formyl, acetyl, trifluoroacetyl, methanesulfonyl, trifluoromethanesulfonyl, ethanesulfonyl, propanesulfonyl, butanesulfonyl, benzenesulfonyl, 4-methylbenzenesulfonyl, and 4-nitrobenzenesulfonyl.

As referred to herein, the term “nucleophile” refers to a group or atom that forms a chemical bond to its reaction partner by donating bonding electrons. Both neutral groups and negatively charged groups or atoms with a free pair of electrons may act as nucleophiles. Useful examples of a nucleophile may include, but are not limited to, azide, bromide, iodide, water, sodium hydroxide, sodium methoxide, sodium ethoxide, hydrogen sulfide, sodium thiohydroxide, sodium thiomethoxide, sodium thioethoxide, ammonia, methyl amine, ethyl amine, sodium phthalimide, urea, and ethyl carbamate.

As referred to herein, the term “nucleophilic substitution reaction” refers to the act of displacing a leaving group L from an sp³-hydridized carbon atom with a nucleophile so as to produce a new chemical bond between the carbon atom and the nucleophile.

As referred to herein, the term “optically pure” refers to a chiral compound that may include more than about 90% of one enantiomer.

As referred to herein, the term “Pramiconazole” refers to (2S-cis)-1-[4-[4-[4-[[4-(2,4-difluorophenyl)-4-(1H-1,2,4-triazol-1-ylmethyl)-1,3-dioxolan-2-yl]methoxy]phenyl]1-piperazinyl]phenyl]-3-(1-methylethyl)-2-imidazolidinone, or as otherwise referred to in the chemical literature as Azoline or R126638.

As referred to herein, the term “substituent” refers to a radical that replaces a hydrogen atom on a hydrocarbon. Useful examples of a radical substituent may include, but are not limited to methyl, ethyl, propyl, butyl, 1-methylethyl, 1,1-dimethylethyl, 1-methylpropyl, 2-methylpropyl, 1-methyl-2-oxopropyl, 2-methylbutyl, cyclopentyl, cyclohexyl, phenyl, -2-C₆H₄CO₂H, -3-C₆H₄CO₂H, -4-C₆H₄CO₂H, —CH₂CO₂H, hydroxy, methoxy, ethoxy, chloro, bromo, and iodo.

What is described herein is a method for manufacture of 2-oxoimidazolidines of Formula I shown below. In Formula I, R₁ represents a radical bonded to the available nitrogen of the corresponding ring and R₂, R₃, R₄, R₅, and R₆ represent radicals bonded to the available carbon atoms on the corresponding ring. Such compounds are useful intermediates in the manufacture of Pramiconazole and other structurally related molecules.

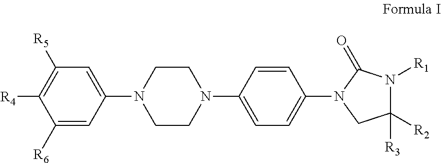

Currently, the 2-oxoimidazolidine of Formula I may be prepared in six processing steps comprised of (i) a reaction of 1-(4-methoxyphenyl)piperazine with 1-chloro-4-nitrobenzene, (ii) a reduction of the nitro group of the product obtained from the first step, (iii) a condensation of the aniline derivative attained in the second step with phenyl chloroformate, (iv) a reaction of the phenylcarbamate acquired from the third step with N-(2,2-dimethoxyethyl)-2-propaneamine, (v) a hydrogenation of the double bond of the 2H-imidazol-2-one intermediate gained from the fourth step, and (vi) a protolytic cleavage of the methoxy group in the product obtained from the fifth step. However, the process described above may have aspects that restrict the utility of this method in large-scale manufacture of 2-oxoimidazolidine, including a time intensive process, a modest overall product yield, a need to separately produce the specialty amine for the fourth step, a potential for trace heavy metal contamination carrying over into the drug substance from the fifth step, and a formation of process impurities that may be difficult to remove and can negatively impact the quality of the drug substance.

Alternatively, a 2-oxoimidazolidine compound of Formula I may also be prepared by the reaction of 1-(4-aminophenyl)-3-(1-methylethyl)-2-oxoimidazolidine with N,N-bis(2-chloro-ethyl)-4-methoxybenzenamine followed by a protolytic cleavage of the methoxy group. The 2-oxoimidazolidine intermediate for this method may be obtained in four additional processing steps consisting of (i) an N-alkylation of 4-nitroaniline with 2-bromoethanamine, (ii) an acylation of the N-aryl-1,2-ethanodiamine attained from the first step with 1,1′-carbonyldiimidazole, (iii) an N-alkylation of the 2-oxoimidazolidine gained from the second step with 1-methylethyl bromide, and (iv) a catalytic reduction of the aryl nitro group in the product achieved from the third step. However, the process described above may have also have certain aspects that may limit the commercial utility of this method, including a lengthy sequence of processing operations, the potential for trace heavy metal contamination carrying over from the fourth step into the drug substance, a modest overall yield, and a high manufacturing cost.

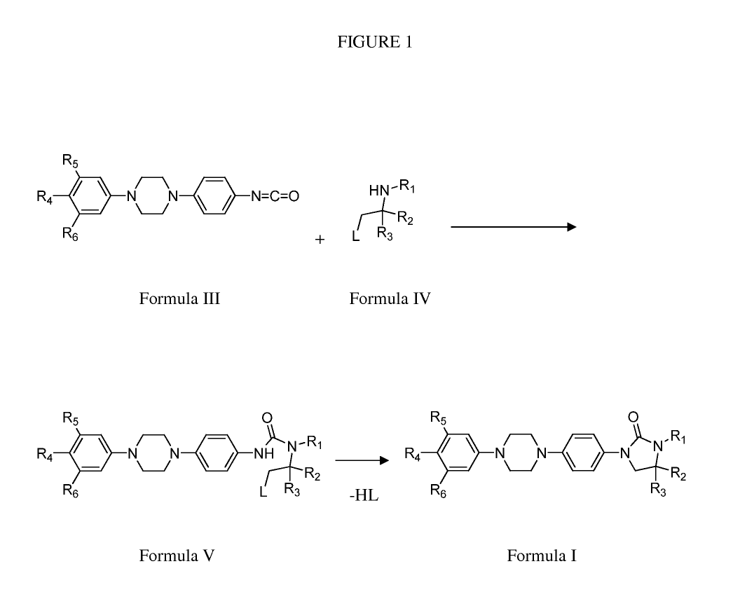

Referring now to FIG. 1, wherein the showings are for purposes of illustrating embodiments of what is described herein and not for purposes of limiting the same, a method for manufacture of 2-oxoimidazolidines is provided. FIG. 1 presents a method for manufacture of 2-oxoimidazolidines of Formula I comprising the steps of: (i) converting an isocyanate and an amine to a urea; and (ii) performing a ring closure of the urea to yield the 2-oxoimidazolidine using the following chemical structures wherein:

the 2-oxoimidazoline is of Formula III;

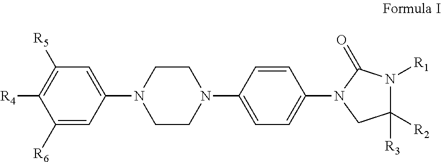

the isocyanate is of Formula III;

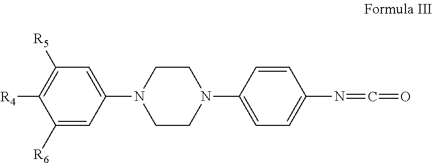

the amine is of Formula IV; and

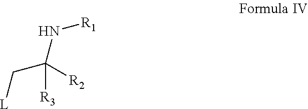

the urea is of Formula V.

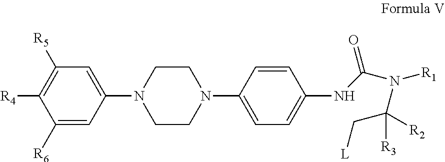

The chemical structures shown above may have substituents selected from the following groups:
R₁ is a radical chosen from methyl, ethyl, propyl, butyl, 1-methylethyl, 1,1-dimethylethyl, 1-methylpropyl, 2-methylpropyl, 1-methyl-2-oxopropyl, 2-methylbutyl, cyclopentyl, cyclohexyl, phenyl, -2-C₆H₄CO₂H, -3-C₆H₄CO₂H, -4-C₆H₄CO₂H, and —CH₂CO₂H;
R₂ and R₃ are radicals chosen from hydrogen, methyl, ethyl, propyl, and 1-methylethyl;
R₄ is a radical chosen from hydrogen, methyl, ethyl, propyl, butyl, 1-methylethyl, 1,1-dimethylethyl; or R₄ is a radical of formula —OR₇ wherein R₇ is a radical chosen from hydrogen, methyl, ethyl, propyl, 1-methylethyl, 1,1-dimethylethyl, 2-propenyl, benzyl, methoxymethyl, ethoxymethyl, methoxyethoxymethyl, and benzyloxymethyl;
R₅ and R₆ are radicals chosen from hydrogen, methyl, ethyl, 1-methylethyl, propyl, butyl, methoxy, ethoxy, n-propoxy, and 1-methylethoxy; and
L is a leaving group chosen from chloro, bromo, iodo, and hydroxy; or L is leaving group of formula —OR₈ wherein R₈ is a radical chosen from formyl, acetyl, trifluoroacetyl, methanesulfonyl, trifluoromethanesulfonyl, ethanesulfonyl, propanesulfonyl, butanesulfonyl, benzenesulfonyl, 4-methylbenzenesulfonyl, and 4-nitrobenzenesulfonyl.

In the first step of the method described herein, a urea of Formula V may be obtained by the reaction of an isocyanate of Formula III with an amine of Formula IV; wherein R₁, R₂, R₃, R₄, R₅, R₆, R₇, and R₈ are defined above, and L is a leaving group that may be chosen from groups listed above. The reaction may be performed by addition of the isocyanate of Formula III, either as a solid or as a solution, to an amine compound of Formula IV. The reaction may be conducted at a temperature between about −25° C. to about 80° C. The reaction may also be conducted at a temperature between about −10° C. to about 35° C. Suitable organic solvents in which to perform the reaction may include, but are not limited to, acetone, acetonitrile; ethereal solvents such as, but not limited to, diethyl ether, di-(1-methylethyl)ether, methyl 1,1-dimethylethyl ether, tetrahydrofuran, and 1,4-dioxane; esters such as, but not limited to, ethyl acetate, n-propyl acetate, and n-butyl acetate; halogenated solvents such as, but not limited to, dichloromethane, chloroform, carbon tetrachloride, 1,2-dichloroethane, and 1,1,2,2-tetrachloroethane; hydrocarbons such as, but not limited to, hexane, heptane, benzene, toluene, xylene, and mesitylene; and mixtures of solvents thereof. In certain instances, the reaction may be performed neat. The urea of Formula V may be isolated by evaporation of the reaction solvent and utilized in the next step without purification. In other instances, the urea of Formula V may be purified by trituration, crystallization, or chromatography.

In a second step of the method described herein, the urea of Formula V may undergo an intramolecular nucleophilic substitution reaction to afford a 2-oxoimidazolidine of Formula I. In this reaction, the nitrogen of the urea may act as a nucleophile to displace the leaving group L from the molecule with the concomitant formation of a new carbon-nitrogen bond. The ring closure reaction may be promoted by the presence of an amine base such as, but not limited to, trimethylamine, triethylamine, tri-n-propylamine, tri-(1-methylethyl)amine, di-(1-methylethyl)ethylamine, tri-n-butylamine, pyridine, N,N-dimethylaminopyridine, and mixtures thereof. The ring closure reaction may also be promoted by the presence of a metal hydroxide of structure M(OH)ₙ, a metal bicarbonate of structure M(HCO₃)ₙ, a metal carbonate of structure MₙCO₃, or a metal alkoxide having the formula M(O-alkyl)ₙ, wherein n may be equal to 1 or 2 and in each case and M may be selected from among an alkali or alkaline earth metal such as, but not limited to, lithium, sodium, potassium, calcium, magnesium, barium, and cesium. In other instances, L may be converted to a group that can be more readily displaced. For example, the conversion of a hydroxy group to an alkyl sulfonate, an aryl sulfonate, or a halide may provide an L group that may be easier to displace. Also, an in situ activation of the hydroxy group under Mitsunobu conditions may provide an L group that may be easier to displace. The selection of a solvent that can achieve a sufficient temperature to drive the nucleophilic substitution reaction to completion can be used. For instance, a polar, aprotic solvent such as, but not limited to, N,N-dimethylformamide, N-methylpyrrolidinone, and dimethyl sulfoxide may be used in a nucleophilic substitution reaction. However, in one embodiment, the solubility of the reaction components may render itself to the use of other solvents, or combinations thereof, that may include, but are not limited to, 1) water; 2) acetone; 3) acetonitrile; 4) alcoholic solvents such as, but not limited to, methanol, ethanol, n-propanol, 1-methylethanol, n-butanol, 1-methylpropanol, 1,1-dimethylethanol, 3-methylbutanol, n-pentanol, and 2,2-dimethylpropanol; 5) ethereal solvents such as, but not limited to, diethyl ether, di-(1-methylethyl)ether, methyl 1,1-dimethylethyl ether, tetrahydrofuran, and 1,4-dioxane; 6) esters such as, but not limited to, ethyl acetate, n-propyl acetate, and n-butyl acetate; 7) halogenated solvents such as, but not limited to, dichloromethane, chloroform, carbon tetrachloride, 1,2-dichloroethane, and 1,1,2,2-tetrachloroethane; and 8) hydrocarbons such as, but not limited to, hexane, heptane, benzene, toluene, xylene, and mesitylene. In another embodiment, the nucleophilic substitution reaction may be performed neat, in a two-phase solvent system, or as a heterogeneous system. In still other instances, the addition of an iodide salt such as, but not limited to, sodium iodide, potassium iodide, and cesium iodide may promote the nucleophilic substitution reaction. The reaction may be conducted at a temperature between about −20° C. to about 120° C. The reaction may also be conducted at a temperature between about −10° C. to about 80° C. The resulting 2-oxoimidazolidine of Formula I may then be purified by trituration, crystallization, or chromatography.

In the method described herein, a bivalent radical may also be formed intramolecularly in certain compounds within the method. In the compounds with R₂ and R₃, including the urea of Formula V or in the 2-oxoimidazolidine of Formula I, both R₂ and R₃ may form a bivalent radical wherein −R₂-R₃— can be chosen from, but not limited to, —CH₂CH₂—, —CH₂CH₂CH₂—, —CH₂(CH₂)₂CH₂—, and —CH₂(CH₂)₃CH₂—. By the formation of bivalent radicals within the method, more complex compounds of the 2-oxoimidazolidine of Formula I may be formed.

In another embodiment of the method described herein, the amine of Formula IV may be comprised of substantially a single enantiomer. In this case, the process may deliver an optically pure 2-oxoimidazolidine of Formula I. When the chiral carbon atom in the amine of Formula IV is that which is within R₁, the chiral center of the 2-oxoimidazolidine of Formula I may retain the same absolute configuration as that of the amine of Formula IV. Furthermore, when the chiral center of a compound of Formula IV is located within a substituent R₁, R₂, or R₃, the stereo-configuration of the group may be preserved in the 2-oxoimidazolidine of Formula I. Therefore, the preparation of a specific enantiomer of the 2-oxoimidazolidine of Formula I may be achieved through proper selection of the optical form of the amine of Formula IV. Moreover, in certain instances, the optical purity of the chiral 2-oxoimidazolidine of Formula I may also be accomplished by crystallization or chiral chromatography.

Pramiconazole may then be produced through a convergent chemical synthesis from (2S-cis)-1-[[2-(bromomethyl)-4-(2,4-difluorophenyl)-1,3-dioxolan-4-yl]methyl]-1H-1,2,4-triazole and the 2-oxoimidazolidine of Formula I. Alternatively, Pramiconazole and certain analogues may be prepared by the reaction of 4-[4-(4-aminophenyl)-1-piperazinyl]phenol with a 1-aryl-2-heteroarylmethyl)-1,3-dioxalan-4-ylmethyl sulfonate or a 2-(halomethyl)-4-(aryl)-1,3-dioxolan-4-yl]methyl]heteroaryl. Thereafter, the 2-oxoimidazolidine ring may be incorporated to form Pramiconazole.

The following examples illustrate the present invention in a way that it can be practiced, but as such these examples should not be interpreted as limitations upon the overall scope of the methods of this invention.

### EXAMPLE 1

### Procedure for Preparation of N-[4-[4-(4-Methoxyphenyl)-1-piperazinyl]-phenyl]-N′-2-methylethyl-N′-[2-hydroxyethyl]urea (Formula Va)

In a dichloromethane (230 mL) solution of 2-(2-methylethylamino)ethanol (210 mmol, 30.9 g) at ambient temperature, 4-[4-(4-methoxyphenyl)-1-piperazinyl]-phenylisocyanate (35 mmol, 10.9 g) was added as a solid over a period of about 40 minutes. During the course of the addition, the solution turned from a red color to a dark red color. Upon completion of the addition, the reaction was stirred for about 2 hours at which time the solution was an opaque brown. The reaction solvent was then evaporated and the residue triturated for about 30 minutes in a mixture of dioxane (140 mL) and water (60 mL). The product was filtered and dried to give N-[4-[4-(4-methoxyphenyl)-1-piperazinyl]-phenyl]-N′-2-methylethyl-N′-[2-hydroxyethyl]urea as a slightly-damp, light brown powder (15.2 g, 100% crude yield), shown below in Structure III.

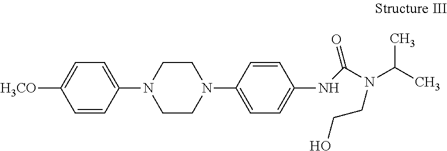 [SMILES: COc1ccc(N2CCN(c3ccc(NC(=O)N(CCO)C(C)C)cc3)CC2)cc1]

### EXAMPLE 2

### Procedure for Preparation of 1-[4-[4-(4-Methoxyphenyl)-1-piperazinyl]phenyl]-3-(1-methylethyl)-2-oxoimidazolidine

To a slurry of N-[4-[4-(4-methoxyphenyl)-1-piperazinyl]-phenyl]-N′-2-methylethyl-N′-[2-hydroxyethyl]urea (35.0 mmol, 14.4 g) in tetrahydrofuran (600 mL) was added potassium tert-butoxide (123.0 mmol, 11.2 g). The reaction vessel was cooled in an ice bath and a tetrahydrofuran (200 mL) solution of p-toluenesulfonyl chloride (57.0 mmol, 10.9 g) was added over a period of 0.5 hours. The resulting beige mixture was allowed to stir at ambient temperature for 14 hours before addition of water (200 mL). The mixture was filtered and the product was washed with water (200 mL) and dried. 1-[4-[4-[4-(4-Methoxyphenyl)-1-piperazinyl]phenyl]-3-(1-methylethyl)-2-oxoimidazolidine, shown below in Structure IV. ¹H NMR (400 MHz, CDCl₃) δ 7.46 (d, J=9.2 Hz, 2H, Ar), 6.96 (d, J=9.2 Hz, 2H, Ar), 6.95 (d, J=9.2 Hz, 2H, Ar), 6.86 (d, J=9.2 Hz, 2H, Ar), 4.30-4.20 (m, 1H, CH), 3.76 (s, 3H, OCH₃), 3.82-3.72 (m, 2H, CH₂), 3.45-3.38 (m, 2H, CH₂), 3.32-3.20 (m, 8H, CH₂).

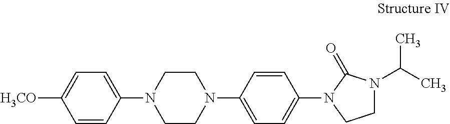 [SMILES: COc1ccc(N2CCN(c3ccc(N4CCN(C(C)C)C4=O)cc3)CC2)cc1]

### EXAMPLE 3

### Procedure for Preparation of 1-[4-[4-(4-Hydroxyphenyl)-1-piperazinyl]phenyl]-3-(1-methylethyl)-2-oxoimidazolidine

1-[4-[4-(4-Methoxyphenyl)-1-piperazinyl]phenyl]-3-(1-methylethyl)-2-oxoi-midazolidine (11.9 mmol, 4.7 g) was heated at reflux in a mixture of 48% hydrobromic acid (30 mL), 33% by weight hydrogen bromide in acetic acid (20 mL) and sodium bisulfite (2.9 mmole, 0.3 g) for about 4 hours. After allowing the reaction to cool to ambient temperature (about 20° C.), water (50 mL) was added and the mixture was stirred for about 30 minutes. The crude product was filtered, transferred into water (50 mL) and neutralized with concentrated ammonium hydroxide solution. Filtration and drying afforded 4.5 grams (100% yield) of the Pramiconazole intermediate of 1-[4-[4-(4-Hydroxyphenyl)-1-piperazinyl]phenyl]-3-(1-methylethyl)-2-oxoimidazolidine shown below in Structure V.

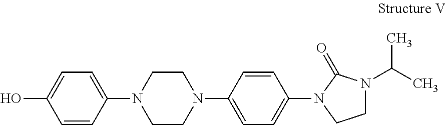 [SMILES: CC(C)N1CCN(c2ccc(N3CCN(c4ccc(O)cc4)CC3)cc2)C1=O]

The implementations have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

I/We claim:

1. A method for preparation of a 2-oxoimidazolidine comprising the steps of:
(i) converting an isocyanate and an amine to a urea; and
(ii) performing a ring closure of said urea to yield said 2-oxoimidazolidine, wherein
said 2-oxoimidazolidine is of Formula I; 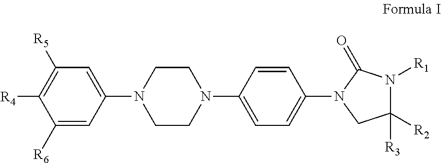
said isocyanate is of Formula III; 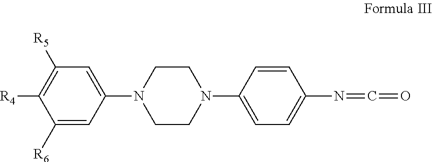
said amine is of Formula IV; 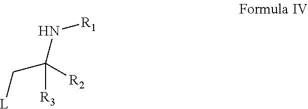
said urea is of Formula V; 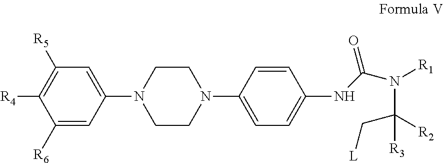
R₁ is a radical chosen from methyl, ethyl, propyl, butyl, 1-methylethyl, 1,1-dimethylethyl, 1-methylpropyl, 2-methylpropyl, 1-methyl-2-oxopropyl, 2-methylbutyl, cyclopentyl, cyclohexyl, phenyl, -2-C₆H₄CO₂H, -3-C₆H₄CO₂H, -4-C₆H₄CO₂H, and —CH₂CO₂H;
R₂ and R₃ are radicals chosen from hydrogen, methyl, ethyl, propyl, and 1-methylethyl;
R₄ is a radical chosen from hydrogen, methyl, ethyl, propyl, butyl, 1-methylethyl, 1,1-dimethylethyl; or R₄ is radical of formula —OR₇ wherein R₇ is a radical chosen from hydrogen, methyl, ethyl, propyl, 1-methylethyl, 1,1-dimethylethyl, 2-propenyl, benzyl, methoxymethyl, ethoxymethyl, methoxyethoxymethyl, and benzyloxymethyl;
R₅ and R₆ are radicals chosen from hydrogen, methyl, ethyl, 1-methylethyl, propyl, butyl, methoxy, ethoxy, n-propoxy, and 1-methylethoxy; and
L is a leaving group chosen from chloro, bromo, iodo, and hydroxy; or L is leaving group of formula —OR₈ wherein R₈ is a radical chosen from formyl, acetyl, trifluoroacetyl, methanesulfonyl, trifluoromethanesulfonyl, ethanesulfonyl, propanesulfonyl, butanesulfonyl, benzenesulfonyl, 4-methylbenzenesulfonyl, and 4-nitrobenzenesulfonyl.

2. The method of claim 1 further comprising the step of:
converting said 2-oxoimidazolidine to Pramiconazole.

3. The method of claim 1, wherein said amine of Formula IV contains a chiral center and is substantially comprised of a single enantiomer to produce said 2-oxoimidazolidine of Formula I in optically pure form.

4. The method of claim 1, wherein said R₂ and said R₃ form a bivalent radical —R₂-R₃— chosen from —CH₂CH₂—, —CH₂CH₂CH₂—, —CH₂(CH₂)₂CH₂— and —CH₂(CH₂)₃CH₂—.
